(19) United States
(12) Patent Application Publication
(10) Pub. No.: US 2025/0339361 A1
(43) Pub. Date: Nov. 6, 2025

(54) COMPOSITIONS COMPRISING XANTHAN GUM AND CRYSTALLINE ALPHA-1,3-GLUCAN

(71) Applicant: Nutrition & Biosciences USA 4, Inc., Rochester, NY (US)

(72) Inventors: Min Zhao, Audubon, PA (US); Karina Esther Aguilar, Wilmington, DE (US); David L. Frattarelli, Wilmington, DE (US); Piera Mattia Pericu, Wilmington, DE (US)

(21) Appl. No.: 19/126,262

(22) PCT Filed: Oct. 31, 2023
(86) PCT No.: PCT/US2023/036374

§ 371 Date: May 1, 2025

Related U.S. Application Data
(60) Provisional application No. 63/382,000, filed on Nov. 2, 2022.

(51) Int. Cl. A61K 8/73; A61Q 19/00

(52) U.S. Cl. CPC A61K 8/73; A61Q 19/00; A61K 2800/5922; A61K 2800/805

(57) ABSTRACT

Aqueous compositions are disclosed herein comprising at least xanthan gum and insoluble alpha-glucan, wherein: (i) at least about 50% of the glycosidic linkages of the insoluble alpha-glucan are alpha-1,3 glycosidic linkages, (ii) the weight-average degree of polymerization of the insoluble alpha-glucan is at least 10, and (iii) the insoluble alpha-glucan is in the form of particles having a degree of crystallinity of at least about 0.65. Further disclosed are methods for producing these compositions, as well as using them in various products.

This application claims the benefit of U.S. Provisional Appl. No. 63/382,000 (filed Nov. 2, 2022), which is incorporated herein by reference in its entirety.

### FIELD

The present disclosure is in the field of polysaccharides. For example, the disclosure pertains to compositions comprising xanthan gum and crystalline alpha-1,3-glucan, and use of this material in various applications.

### BACKGROUND

Driven by a desire to use polysaccharides in various applications, researchers have explored for polysaccharides that are biodegradable and that can be made economically from renewably sourced feedstocks. One such polysaccharide is alpha-1,3-glucan, an insoluble glucan polymer characterized by having alpha-1,3-glycosidic linkages. This polymer has been prepared, for example, using a glucosyltransferase enzyme isolated from Streptococcus salivarius (Simpson et al., Microbiology 141:1451-1460, 1995). Also for example, U.S. Pat. No. 7,000,000 disclosed the preparation of a spun fiber from enzymatically produced alpha-1,3-glucan. Various other glucan materials have also been studied for developing new or enhanced applications. For example, U.S. Patent Appl. Publ. No. 2015/0232819 discloses enzymatic synthesis of several insoluble glucans having mixed alpha-1,3 and -1,6 linkages.

New compositions comprising alpha-1,3-glucan are desired to enhance the economic value and performance characteristics of this material in various applications. Compositions are described herein comprising xanthan gum and crystalline alpha-1,3-glucan, for example, that address this need.

### SUMMARY

In one embodiment, the present disclosure concerns an aqueous composition comprising xanthan gum and insoluble alpha-glucan, wherein:
(i) at least about 50% of the glycosidic linkages of the insoluble alpha-glucan are alpha-1,3 glycosidic linkages,
(ii) the weight-average degree of polymerization (DPw) of the insoluble alpha-glucan is at least 10, and
(iii) the insoluble alpha-glucan is in the form of particles having a degree of crystallinity of at least about 0.65.

In another embodiment, the present disclosure concerns a method of producing an aqueous composition as presently disclosed, wherein the method comprises: blending together at least water, xanthan gum, and insoluble alpha-glucan herein.

### BRIEF DESCRIPTION OF THE DRAWINGS

FIG. 1: Shown are viscosity measurements for various aqueous preparations of MCG and xanthan gum over a range of shear rates. This figure shows the tightness of the viscosity read-outs for the six different samples at each selected shear rate. Given the black-and-white format of this figure, it is not intended to show specific viscosity values for each sample. Refer to the Examples.

FIG. 2: Shown are personal care serums with 0.4 wt % MCG and 0.2 wt % xanthan gum as thickeners (Example 6), or with only 0.2 wt % xanthan gum as thickener (Control 4). Refer to the Examples.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like. The numerical values of the various ranges in the present disclosure, unless expressly indicated otherwise, are stated as approximations as though the minimum and maximum values within the stated ranges were both proceeded by the word “about”. In this manner, slight variations above and below the stated ranges can typically be used to achieve substantially the same results as values within the ranges. Also, the disclosure of these ranges is intended as a continuous range including each and every value between the minimum and maximum values.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

It is to be appreciated that certain features of the present disclosure, which are, for clarity, described above and below in the context of aspects/embodiments, may also be provided in combination in a single element. Conversely, various features of the disclosure that are, for brevity, described in the context of a single aspect/embodiment, can also be provided separately or in any sub-combination.

The terms “alpha-glucan”, “alpha-glucan polymer” and the like are used interchangeably herein. An alpha-glucan is a polymer comprising glucose monomeric units linked together by alpha-glycosidic linkages. In typical aspects, the glycosidic linkages of an alpha-glucan herein are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of an alpha-glucan polymer herein include alpha-1,3-glucan.

The terms “alpha-1,3-glucan”, “poly alpha-1,3-glucan”, “alpha-1,3-glucan polymer” and the like are used interchangeably herein. Alpha-1,3-glucan is an alpha-glucan comprising glucose monomeric units linked together by glycosidic linkages, wherein at least about 50% of the glycosidic linkages are alpha-1,3. Alpha-1,3-glucan in some aspects comprises about, or at least about, 90%, 95%, or 100% alpha-1,3 glycosidic linkages. Most or all of the other linkages, if present, in alpha-1,3-glucan herein typically are alpha-1,6, though some linkages may also be alpha-1,2 and/or alpha-1,4. Alpha-1,3-glucan herein is typically water-insoluble.

The terms “xanthan gum”, “xanthan”, “xanthan polysaccharide” and like terms herein refer to a water-soluble polysaccharide that typically is produced by Xanthomonas bacteria (e.g., X. campestris), such as in an industrial fermentation. Xanthan gum can be as disclosed in, for example, Biopolymers for Food Design (A. M. Grumezescu and A. M. Holban, Ed., Chapter 4, N. Jindal and J. S. Khattar, Microbial Polysaccharides in Food Industry, Academic Press, 2018) or U.S. Patent Appl. Publ. No. 2003/0143179, which are incorporated herein by reference.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages) and 1,3-alpha-D-glycosidic linkages (herein also referred to as “alpha-1,3” linkages).

The glycosidic linkage profile of a polysaccharide can be determined using any method known in the art. For example, a linkage profile can be determined using methods using nuclear magnetic resonance (NMR) spectroscopy (e.g., ¹³C NMR and/or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The “molecular weight” of a polysaccharide herein such as an alpha-glucan can be represented as weight-average molecular weight (Mw) or number-average molecular weight (Mn), the units of which are in Daltons (Da) or grams/mole. Alternatively, molecular weight can be represented as DPw (weight average degree of polymerization) or DPn (number average degree of polymerization). The molecular weight of smaller polysaccharide polymers such as oligosaccharides can optionally be provided as “DP” (degree of polymerization), which simply refers to the number of monomers comprised within the polysaccharide; “DP” can also characterize the molecular weight of a polymer on an individual molecule basis. Various means are known in the art for calculating these various molecular weight measurements such as with high-pressure liquid chromatography (HPLC), size exclusion chromatography (SEC), or gel permeation chromatography (GPC).

As used herein, Mw can be calculated as Mw=ΣNiMi²/ΣNiMi; where Mi is the molecular weight of an individual chain i and Ni is the number of chains of that molecular weight. Besides SEC, the Mw of a polymer can be determined by other techniques such as static light scattering, mass spectrometry, MALDI-TOF (matrix-assisted laser desorption/ionization time-of-flight), small angle X-ray or neutron scattering, or ultracentrifugation. As used herein, Mn can be calculated as Mn=ΣNiMi/ΣNi where Mi is the molecular weight of a chain i and Ni is the number of chains of that molecular weight. Besides SEC, the Mn of a polymer can be determined by various colligative property methods such as vapor pressure osmometry, end-group determination by spectroscopic methods such as proton NMR, proton FTIR, or UV-Vis. As used herein, DPn and DPw can be calculated from Mn and Mw, respectively, by dividing them by molar mass of the one monomer unit Mi. In the case of unsubstituted glucan polymer, M₁=162. In the case of a substituted (derivatized) glucan polymer, M₁=162+M_{f}×DoS, where M_{f} is molar mass of the substituting group, and DoS is degree of substitution (average number of substituted groups per one glucose unit of the glucan polymer).

The terms “crystalline”, “crystalline solid”, “crystal” and like terms herein refer to a solid material whose constituents are arranged in a regularly ordered structure forming a lattice; such material typically is a portion of a larger composition having both crystalline and amorphous regions. An “amorphous” material is non-crystalline in that its constituents are not organized in a definite lattice pattern, but rather are randomly organized. Crystalline materials, but not amorphous materials, usually have a characteristic geometric shape (e.g., plate). The terms “crystallinity”, “crystallinity index” (CI), “degree of crystallinity” and the like herein refer to the fractional amount (mass fraction or volume fraction) of an insoluble alpha-glucan that is crystalline, and can be referred to in decimal or percentage form (e.g., a crystallinity of 0.65 corresponds to a crystallinity of 65%). This fractional amount is of a total amount or volume that includes the amorphous content of the insoluble alpha-glucan. Crystallinity herein can be as measured using techniques such as differential scanning calorimetry (DSC), X-ray diffraction (XRD), small angle X-ray scattering (SAXS), infrared spectroscopy, and/or density measurements according to, for example, Struszczyk et al. (1987, J. Appl. Polym. Sci. 33:177-189), U.S. Patent Appl. Publ. Nos. 2015/0247176, 2010/0233773, 2015/0152196, 2020/0181370, or 2021/0130504, which are all incorporated herein by reference. In some aspects, the crystallinity of insoluble alpha-glucan herein can be as determined according to the methodology disclosed in the below Examples (Materials/Methods).

The terms “particle”, “particulate” and like terms are interchangeably used herein, and refers to the smallest identifiable unit in a particulate system. The term “particulated” and like terms can be used to characterize particles of insoluble alpha-glucan herein; particulated insoluble alpha-glucan in typical aspects of the present disclosure is as this material exists when dispersed under aqueous conditions. Particle size in some aspects can refer to particle diameter and/or the length of the longest particle dimension. The average size can be based on the average of diameters and/or longest particle dimensions of at least 50, 100, 500, 1000, 2500, 5000, or 10000 or more particles, for example. Particles herein can be in plate form, for instance. Particle size herein can be measured by a process comprising light scattering or electrical impedance change (e.g., using a Coulter Counter), for example, such as described in any of U.S. Pat. Nos. 6,091,492, 6,741,350, or 9297737 (each incorporated herein by reference). Particle size and/or distributions can be as measured for particles comprised in an aqueous dispersion, for example. Particle size herein can optionally be expressed by a “D₁₀”, “D₅₀”, “D₉₀”, etc. value; for example, a D₅₀ value is the diameter for which 50% by weight of the particles in a composition (e.g., dispersion) have a diameter under that diameter, and 50% by weight of the particles have a diameter greater than that diameter.

The terms “plate”, “platy”, “plate-like”, “flakey” and like terms herein characterize the shape of insoluble alpha-glucan particles in some aspects. Particles having this shape herein generally are flat (more two-dimensional than three-dimensional), as opposed to being spherical, cylindrical, fibrillar, fibrous, rod-like, cubic, acicular, spongey/porous, lamellar, or of some other shape. Particles herein can optionally be referred to as “plates”, “platelets”, and like terms, and/or collectively as “microcrystalline glucan” (MCG) and like terms.

A composition herein that is “dry” or “dried” typically has less than 12, 11, 10, 9, 8, 7, 6, 5, 4, 3, 2, 1, 0.5, or 0.1 wt % water comprised therein.

The terms “aqueous liquid”, “aqueous fluid”, “aqueous conditions”, “aqueous reaction conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some aspects. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., colloidal dispersions), suspensions and emulsions, for example.

As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. All, or a portion of, the particles of a colloidal dispersion such as a hydrocolloid can comprise insoluble alpha-glucan as presently disclosed. The terms “dispersant” and “dispersion agent” are used interchangeably herein to refer to a material that promotes the formation and/or stabilization of a dispersion. “Dispersing” herein refers to the act of preparing a dispersion of a material in an aqueous liquid. As used herein, the term “latex” (and like terms) refers to a dispersion of one or more types of polymer particles in water or aqueous solution; typically, at least particles herein are in a latex composition as a dispersed polymer component. In some aspects, a latex is an emulsion that comprises a dispersion of at least particles herein. An “emulsion” herein is a dispersion of minute droplets of one liquid in another liquid in which the droplets are not soluble or miscible (e.g., a non-polar substance such as oil or other organic liquid such as an alkane, in a polar liquid such as water or aqueous solution). An emulsion can further comprise dispersed alpha-glucan herein, for example, which optionally can stabilize the emulsion. In some aspects, however, an emulsion herein can be a “dry emulsion”. A dry emulsion is typically produced by removing all or most (e.g. >95%, >99%, or >99.5%) of the water of a liquid emulsion, such as by freeze-drying or spray-drying.

Compositions of the present disclosure can provide stability to a dispersion or emulsion, for example. The “stability” (or the quality of being “stable”) of a dispersion or emulsion herein is, for example, the ability of dispersed particles of a dispersion, or liquid droplets dispersed in another liquid (emulsion), to remain dispersed (e.g., about, or at least about, 70, 75, 80, 85, 90, 95, 96, 97, 98, 99, or 100 wt % of the particles of the dispersion or liquid droplets of the emulsion are in a dispersed state) for a period of about, or at least about, 2, 4, 6, 9, 12, 18, 24, 30, or 36 months following initial preparation of the dispersion or emulsion. A stable dispersion or emulsion in some aspects can resist total sedimentation, flocculation, and/or coalescence of dispersed/emulsified material.

An alpha-glucan that is “insoluble”, “aqueous-insoluble”, “water-insoluble” (and like terms) (e.g., alpha-1,3-glucan with a DP of 8 or higher) herein does not dissolve (or does not appreciably dissolve) in water or other aqueous conditions, optionally where the aqueous conditions are further characterized to have a pH of 4-9 (e.g., pH 6-8) and/or temperature of about 1 to 130° C. (e.g., 20-25° C.). In some aspects, less than 1.0 gram (e.g., no detectable amount) of an aqueous-insoluble alpha-glucan herein dissolves in 1000 milliliters of such aqueous conditions (e.g., water at 23° C.). In contrast, glucans such as certain oligosaccharides herein that are “soluble”, “aqueous-soluble”, “water-soluble” and the like (e.g., alpha-1,3-glucan with a DP less than 8) appreciably dissolve under these conditions.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa-s), for example. A centipoise is one one-hundredth of a poise; one poise is equal to 0.100 kg·m⁻¹·s⁻¹.

The terms “household care product”, “home care product”, and like terms typically refer to products, goods and services relating to the treatment, cleaning, caring, and/or conditioning of a home and its contents. The foregoing includes, for example, chemicals, compositions, products, or combinations thereof having application in such care.

A “fabric care composition”, “laundry care composition”, and like terms refer to any composition suitable for treating fabric, non-wovens, and/or any similar material in some manner. Examples of such a composition include laundry detergents and fabric softeners.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The term “personal care product” and like terms typically refer to products, goods and services relating to the treatment, cleaning, cleansing, caring or conditioning of a person. The foregoing include, for example, chemicals, compositions, products, or combinations thereof having application in such care.

The term “medical product” and like terms typically refer to products, goods and services relating to the diagnosis, treatment, and/or care of patients.

The term “industrial product” and like terms typically refer to products, goods and services used in industrial or institutional settings, but typically not by individual consumers.

The terms “ingestible product”, “ingestible composition” and the like refer to any substance that, either alone or together with another substance, may be taken orally (i.e., by mouth), whether intended for consumption or not. Thus, an ingestible product includes food/beverage products. “Food/beverage products” refer to any edible product intended for consumption (e.g., for nutritional purposes) by humans or animals, including solids, semi-solids, or liquids. A “food” herein can optionally be referred to as a “foodstuff”, “food product”, or other like term, for example. “Non-edible products” (“non-edible compositions”) refer to any composition that can be taken by the mouth for purposes other than food or beverage consumption. Examples of non-edible products herein include supplements, nutraceuticals, functional food products, pharmaceutical products, oral care products (e.g., dentifrices, mouthwashes), and cosmetic products such as sweetened lip balms. A “pharmaceutical product”, “medicine”, “medication”, “drug” or like term herein refers to a composition used to treat disease or injury, and can be administered enterally or parenterally.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The term “isolated” means a substance (or process) in a form or environment that does not occur in nature. A non-limiting example of an isolated substance includes any aqueous alpha-glucan composition disclosed herein (e.g., further comprising xanthan gum). It is believed that the embodiments disclosed herein are synthetic/man-made (could not have been made or practiced except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some aspects of the present disclosure concern an aqueous composition (product) comprising xanthan gum and insoluble alpha-glucan, wherein:
(i) at least about 50% of the glycosidic linkages of the insoluble alpha-glucan are alpha-1,3 glycosidic linkages,
(ii) the weight-average degree of polymerization (DPw) of the insoluble alpha-glucan is at least 10, and
(iii) the insoluble alpha-glucan is in the form of particles having a degree of crystallinity of at least about 0.65.

Such compositions as presently disclosed have several advantageous features. For example, aqueous compositions herein can exhibit synergistic enhancement of viscosity. Also for example, aqueous compositions herein can exhibit enhanced stability.

An aqueous composition of the present disclosure comprises insoluble alpha-glucan, wherein at least about 50% of the glycosidic linkages of the insoluble alpha-glucan are alpha-1,3 glycosidic linkages. In some aspects, about, or at least about, 50%, 60%, 70%, 80%, 85%, 90%, 91%, 92%, 93%, 94%, 95%, 96%, 97%, 98%, 99%, 99.5%, or 100% of the glycosidic linkages of insoluble alpha-glucan are alpha-1,3 glycosidic linkages. Typically, the glycosidic linkages that are not alpha-1,3 are mostly or entirely alpha-1,6. It should be understood that the higher the percentage of alpha-1,3 linkages present in an insoluble alpha-glucan, the greater the probability that the glucan is linear, since there are lower occurrences of certain linkages that might be part of branch points. In some aspects, insoluble alpha-glucan has no branch points or less than about 5%, 4%, 3%, 2%, or 1% branch points as a percent of the glycosidic linkages in the alpha-glucan.

In some aspects, the DPw, DPn, or DP of insoluble alpha-glucan can be about, at least about, or less than about, 10, 15, 20, 25, 30, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 125, 150, 175, 200, 15-100, 25-100, 35-100, 15-80, 25-80, 35-80, 15-60, 25-60, 35-60, 15-55, 25-55, 25-50, 35-55, 35-50, 35-45, 35-40, 40-100, 40-80, 40-60, 40-55, 40-50, 45-60, 45-55, or 45-50.

An aqueous composition of the present disclosure can, in some aspects, comprise insoluble alpha-glucan that is in the form of particles having a degree of crystallinity of at least about 0.65. The degree of crystallinity (or crystallinity index [CI]) of insoluble alpha-glucan particles herein can be about, or at least about, 0.55, 0.60, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.60-0.83, 0.65-0.83, 0.67-0.83, 0.69-0.83, 0.60-0.81, 0.65-0.81, 0.67-0.81, 0.69-0.81, 0.60-0.78, 0.65-0.78, 0.67-0.78, 0.69-0.78, 0.60-0.76, 0.65-0.76, 0.67-0.76, or 0.69-0.76, for example. In general, that portion of insoluble alpha-glucan herein that is not crystalline is amorphous. Flowing from the foregoing crystallinity values, the wt % of particles that is amorphous is about, or less than about, 45%, 40%, 35%, 30%, 25%, 20%, or 15%, for example. The degree of crystallinity of alpha-glucan particles herein can be as when measured according to any suitable method, such as follows. A sample of insoluble alpha-glucan herein is dried for at least about 2 hours (e.g., 8-12 hours) in a vacuum oven set at about 55-65° C. (e.g., 60° C.). The sample is then be packed into a stainless steel holder with a well of about 1-2 cm wide by 3-5 cm long by 3-5 mm deep, after which the holder is loaded into a suitable diffractometer (e.g., X'PERT MPD POWDER diffractometer, PANalytical B.V., The Netherlands) set in reflection mode to measure the X-ray diffraction pattern of the sample. The X-ray source is a Cu X-ray tube line source with an optical focusing mirror and a ˜ 1/16° narrowing slit. X-rays are detected with a 1-D detector and an anti-scatter slit set at ˜⅛°. Data are collected in the range of about 4 to 60 degrees of two-theta at about 0.1 degrees per step. The resulting X-ray pattern is then analyzed by subtracting a linear baseline from about 7.2 to 30.5 degrees, subtracting the XRD pattern of a known amorphous alpha-1,3-glucan sample that has been scaled to fit the data, and then fitting the remaining crystal peaks in that range with a series of Gaussian curves corresponding to known dehydrated alpha-1,3-glucan crystal reflections. The area corresponding to the crystal peaks is then divided by the total area under the baseline-subtracted curve to yield a crystallinity index.

At least about 80 wt % of particles of insoluble alpha-glucan having any of the foregoing degrees of crystallinity can be in the form of plates, for example. In some aspects, about, or at least about, 60, 65, 70, 75, 80, 81, 82, 83, 84, 85, 86, 87, 88, 89, 90, 91, 92, 93, 94, 95, 96, 97, 98, 99, 60-85, 60-80, 60-75, 60-70, 65-85, 65-80, 65-75, 65-70, 70-85, 70-80, or 70-75 wt % of the particles of insoluble alpha-glucan are in the form of plates. Plates of insoluble alpha-glucan herein can be visually appreciated when viewed by electron microscopy such as TEM or SEM, for example. Typically, the balance of particles of insoluble alpha-glucan are of non-plate form. In some aspects, the balance of the particles that are of non-plate form can be characterized as being fibrillar and/or striated in appearance. However, in some aspects, about, or at least about, 10, 20, 30, 40, 50, 60, or 70 wt % of the particles of insoluble alpha-glucan in a composition herein are in the form of plates.

In some aspects, at least about 65% by weight of insoluble alpha-glucan particles having any of the foregoing degrees of crystallinity have a diameter of less than 1.0 micron. Yet, in some aspects, about, or at least about, 65%, 70%, 75%, 80%, 85%, 90%, 95%, 65-95%, 70-95%, 75-95%, 80-95%, 85-95%, 65-90%, 70-90%, 75-90%, 80-90%, 85-90%, 65-85%, 70-85%, 75-85%, or 80-85% by weight of insoluble alpha-glucan particles have a diameter of less than about 1.0 micron. In some aspects, about 40-60%, 40-55%, 45-60%, 45-55%, 47-53%, 48-52%, 49-51%, or 50% by weight of the insoluble alpha-glucan particles have a diameter of about, or less than about, 1.0, 0.9, 0.8, 0.7, 0.6, 0.5, 0.4, 0.35, 0.34, 0.32, 0.30, 0.28, 0.26, 0.25, 0.24, 0.23, 0.22, 0.21, 0.20, 0.19, 0.18, 0.17, 0.16, 0.15, 0.14, 0.13, 0.12, 0.11, 0.10, 0.10-1.0, 0.10-0.80, 0.10-0.60, 0.10-0.40, 0.10-0.35, 0.10-0.30, 0.10-0.25, 0.10-0.20, 0.15-0.35, 0.15-0.30, 0.15-0.25, or 0.15-0.20 micron. In some aspects, about 40-60%, 40-55%, 45-60%, 45-55%, 47-53%, 48-52%, 49-51%, or 50% by weight of insoluble alpha-glucan particles are aggregates of the foregoing smaller diameter particles, and have a diameter of about, less than about, or at least about, 10, 25, 50, 100, 150, 200, 250, 300, 350, 400, 450, 500, 550, 600, 10-600, 10-550, 10-500, 50-600, 50-550, 50-500, 100-600, 100-550, 100-500, 150-600, 150-550, 150-500, 200-600, 200-550, 200-500, 250-600, 250-550, or 250-500 microns. Alpha-glucan particles having any of the foregoing degrees of crystallinity can have a thickness of about 0.010, 0.015, 0.020, 0.025, 0.030, or 0.010-0.030 micron, for example; such a thickness can optionally be in conjunction with any of the foregoing diameter aspects. The foregoing particle size and/or distributions for crystalline particles herein can be as measured for particles comprised in an aqueous dispersion, and/or as measured using a light scatter technique, for example.

Insoluble alpha-glucan can be in the form of particles in some aspects. As comprised in an aqueous composition herein such as a dispersion or emulsion, about 40-60%, 40-55%, 45-60%, 45-55%, 47-53%, 48-52%, 49-51%, or 50% by weight of insoluble alpha-glucan particles herein can have a diameter (i.e., D₅₀) of about, less than about, or at least about, 1, 2, 3, 4, 5, 6, 7, 8, 9, 10, 11, 12, 13, 14, 15, 16, 17, 18, 19, 20, 21, 22, 23, 24, 25, 1-25, 1-22, 1-20, 1-18, 5-25, 5-22, 5-20, 5-18, 15-22, 15-20, 15-18, 16-22, 16-20, or 16-18 microns, for example.

Insoluble alpha-glucan in the form of particles having a degree of crystallinity of at least about 0.65 can be produced, for example, by a method comprising the following steps: (a) providing insoluble alpha-glucan (precursor) as produced in an enzymatic reaction comprising at least water, sucrose and a glucosyltransferase enzyme that synthesizes the insoluble alpha-glucan, wherein the insoluble alpha-glucan has a DPw or DPn of at least about, or over about, 100, 150, or 200, and at least 50% of its glycosidic linkages are alpha-1,3 glycosidic linkages; (b) hydrolyzing the insoluble alpha-glucan (precursor) to insoluble alpha-glucan particles with a DPw or DPn, for example, of about 10 to 100 (e.g., any DPw or DPn value herein falling in this range), wherein the hydrolyzing is performed under aqueous conditions at a pH of 2.0 or less, and (c) optionally isolating the insoluble alpha-glucan particles produced in step (b). Step (b) of this method can optionally be characterized as an “acid hydrolysis” method or reaction. Insoluble alpha-glucan precursor herein for entry into acid hydrolysis is itself insoluble alpha-glucan, but has a molecular weight that is greater than that of the insoluble alpha-glucan produced by the hydrolysis method. An insoluble alpha-glucan precursor can have a glycosidic linkage profile as disclosed above (e.g., at least about 50%, 60%, 70%, 80%, 90%, 95%, 99%, 99.5%, or 100% alpha-1,3 glycosidic linkages) and a DPw or DPn of about, at least about, or over about, 200 (e.g., any such DPw or DPn as disclosed above).

Insoluble alpha-glucan herein can be as disclosed (e.g., molecular weight, linkage profile, crystallinity, and/or production method), for example, in U.S. Pat. Nos. 7,000,000, 8,871,474, 10,301,604, or 10,260,053, or U.S. Patent Appl. Publ. Nos. 2019/0112456, 2019/0078062, 2019/0078063, 2018/0340199, 2018/0021238, 2018/0273731, 2017/0002335, 2015/0232819, 2015/0064748, 2020/0165360, 2020/0131281, 2019/0185893, 2019/0276806, or 2021/0130504, which are each incorporated herein by reference. Insoluble alpha-glucan can be produced, for example, by an enzymatic reaction comprising at least water, sucrose and a glucosyltransferase enzyme that synthesizes the insoluble alpha-glucan. Glucosyltransferases, reaction conditions, and/or processes contemplated to be useful for producing insoluble alpha-glucan can be as disclosed in any of the foregoing references.

In some aspects, a glucosyltransferase enzyme for producing an insoluble alpha-glucan can comprise an amino acid sequence that is 100% identical to, or at least 90%, 91%, 92%, 93%, 94%, 95%, 96%, 97%, 98%, 98.5%, 99%, or 99.5% identical to, SEQ ID NO:2, 4, 6, 8, 10, 12, 14, 16, 18, 20, 26, 28, 30, 34, or 59, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20, and have glucosyltransferase activity; these amino acid sequences are disclosed in U.S. Patent Appl. Publ. No. 2019/0078063, which is incorporated herein by reference. It is noted that a glucosyltransferase enzyme comprising SEQ ID NO:2, 4, 8, 10, 14, 20, 26, 28, 30, 34, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20, can synthesize insoluble alpha-glucan comprising at least about 90% (˜100%) alpha-1,3 linkages.

Insoluble alpha-glucan herein typically does not have any chemical derivatization (e.g., etherification, esterification, phosphorylation, sulfation, oxidation, carbamation) (e.g., no substitution of hydrogens of glucan hydroxyl groups with a non-sugar chemical group). However, in some aspects, insoluble alpha-glucan can be a charged (e.g., cationic or anionic) derivative of an alpha-glucan as disclosed herein. The DoS of such a derivative typically is less than about 0.3, 0.25, 0.2, 0.15, 0.1, or 0.05. The type of derivative can be any of the derivatives disclosed herein (e.g., ether, ester). Typically, insoluble alpha-glucan herein is enzymatically derived in an inert vessel (typically under cell-free conditions) and is not derived from a cell wall (e.g., fungal cell wall).

In some aspects, an aqueous composition of the present disclosure comprises about, at least about, or less than about, 0.05%, 0.1%, 0.2%, 0.3%, 0.4%, 0.5%, 0.75%, 1%, 1.5%, 2%, 2.5%, 3%, 4%, 5%, 0.05-5%, 0.05-3%, 0.05-2%, 0.05-1.5%, 0.05-1%, 0.05-0.75%, 0.05-0.5%, 0.05-0.4%, 0.05-0.3%, 0.1-5%, 0.1-3%, 0.1-2%, 0.1-1.5%, 0.1-1%, 0.1-0.75%, 0.1-0.5%, 0.1-0.4%, 0.1-0.3%, 0.2-5%, 0.2-3%, 0.2-2%, 0.2-1.5%, 0.2-1%, 0.2-0.75%, 0.2-0.5%, 0.2-0.4%, 0.3-5%, 0.3-3%, 0.3-2%, 0.3-1.5%, 0.3-1%, 0.3-0.75%, 0.3-0.5%, 0.3-0.4%, 0.5-5%, 0.5-3%, 0.5-2%, 0.5-1.5%, 0.5-1%, 0.5-0.75%, 0.75-5%, 0.75-3%, 0.75-2%, 0.75-1.5%, 0.75-1%, 1-5%, 1-3%, 1-2%, or 1-1.5% by weight of insoluble alpha-glucan herein.

In some aspects, an aqueous composition of the present disclosure comprises about, at least about, or less than about, 0.05%, 0.1%, 0.2%, 0.3%, 0.4%, 0.5%, 0.75%, 1%, 1.5%, 2%, 0.05-2%, 0.05-1.5%, 0.05-1%, 0.05-0.75%, 0.05-0.5%, 0.05-0.4%, 0.05-0.3%, 0.1-1%, 0.1-0.75%, 0.1-0.5%, 0.1-0.4%, 0.1-0.3%, 0.2-1%, 0.2-0.75%, 0.2-0.5%, 0.2-0.4%, 0.3-1%, 0.3-0.75%, 0.3-0.5%, 0.3-0.4%, 0.5-1%, or 0.5-0.75% by weight of xanthan gum herein.

In some aspects, an aqueous composition of the present disclosure comprises about, or at least about, 50%, 60%, 70%, 80%, 90%, 92.5%, 95%, 96%, 97%, 98%, 99%, 50-60%, 50-70%, 50-80%, 50-90%, 50-95%, 50-97%, 60-70%, 60-80%, 60-90%, 60-95%, 60-97%, 70-80%, 70-90%, 70-95%, 70-97% 80-90%, 80-95%, 80-97%, 80-95%, or 90-97% by weight water.

Any combination of wt % values/ranges for (i) xanthan gum, (ii) insoluble alpha-glucan, and (iii) water as presently disclosed can characterize an aqueous composition herein. Merely as examples, an aqueous composition herein can comprise at least:
(A) (i) 0.1-1.0 wt %, 0.2-1.0 wt %, or 0.3-1.0 wt % xanthan gum, and (ii) 0.1-1.0 wt %, 0.2-1.0 wt %, or 0.3-1.0 wt % insoluble alpha-glucan;
(B) (i) 0.1-0.75 wt %, 0.2-0.75 wt %, or 0.3-0.75 wt % xanthan gum, and (ii) 0.1-0.75 wt %, 0.2-0.75 wt %, or 0.3-0.75 wt % insoluble alpha-glucan;
(C) (i) 0.1-0.5 wt %, 0.2-0.5 wt %, or 0.3-0.5 wt % xanthan gum, and (ii) 0.1-0.5 wt %, 0.2-0.5 wt %, or 0.3-0.5 wt % insoluble alpha-glucan;
(D) (i) 0.1-0.3 wt % or 0.2-0.3 wt % xanthan gum, and (ii) 0.1-1.0 wt %, 0.2-1.0 wt %, or 0.3-1.0 wt % insoluble alpha-glucan;
(E) (i) 0.1-0.3 wt % or 0.2-0.3 wt % xanthan gum, and (ii) 0.1-0.75 wt %, 0.2-0.75 wt %, or 0.3-0.75 wt % insoluble alpha-glucan;
(F) (i) 0.1-1.0 wt %, 0.2-1.0 wt %, or 0.3-1.0 wt % xanthan gum, and (ii) 0.5-5 wt %, 0.5-4 wt %, 0.5-3 wt %, 0.5-2 wt %, 1-5 wt %, 1-4 wt %, 1-3 wt %, 1-2 wt %, 1.5-5 wt %, 1.5-4 wt %, 1.5-3 wt %, 1.5-2 wt %, 2-5 wt %, 2-4 wt %, or 2-3 wt % insoluble alpha-glucan;
(G) (i) 0.1-0.75 wt %, 0.2-0.75 wt %, or 0.3-0.75 wt % xanthan gum, and (ii) 0.5-5 wt %, 0.5-4 wt %, 0.5-3 wt %, 0.5-2 wt %, 1-5 wt %, 1-4 wt %, 1-3 wt %, 1-2 wt %, 1.5-5 wt %, 1.5-4 wt %, 1.5-3 wt %, 1.5-2 wt %, 2-5 wt %, 2-4 wt %, or 2-3 wt % insoluble alpha-glucan;
(H) (i) 0.1-0.5 wt %, 0.2-0.5 wt %, or 0.3-0.5 wt % xanthan gum, and (ii) 0.5-5 wt %, 0.5-4 wt %, 0.5-3 wt %, 0.5-2 wt %, 1-5 wt %, 1-4 wt %, 1-3 wt %, 1-2 wt %, 1.5-5 wt %, 1.5-4 wt %, 1.5-3 wt %, 1.5-2 wt %, 2-5 wt %, 2-4 wt %, or 2-3 wt % insoluble alpha-glucan;
(I) (i) 0.1-0.3 wt % or 0.2-0.3 wt % xanthan gum, and (ii) 0.5-5 wt %, 0.5-4 wt %, 0.5-3 wt %, 0.5-2 wt %, 1-5 wt %, 1-4 wt %, 1-3 wt %, 1-2 wt %, 1.5-5 wt %, 1.5-4 wt %, 1.5-3 wt %, 1.5-2 wt %, 2-5 wt %, 2-4 wt %, or 2-3 wt % insoluble alpha-glucan; or
(J) (i) 0.1-0.3 wt % or 0.2-0.3 wt % xanthan gum, and (ii) 0.5-5 wt %, 0.5-4 wt %, 0.5-3 wt %, 0.5-2 wt %, 1-5 wt %, 1-4 wt %, 1-3 wt %, 1-2 wt %, 1.5-5 wt %, 1.5-4 wt %, 1.5-3 wt %, 1.5-2 wt %, 2-5 wt %, 2-4 wt %, or 2-3 wt % insoluble alpha-glucan.
   A composition comprising any one of (A)-(J) can comprise any of the foregoing levels of water, for example.

Typically, one or more additional components/ingredients can be present in an aqueous composition herein comprising (i) xanthan gum, (ii) insoluble alpha-glucan, and (iii) water. Examples of other components can be any as disclosed herein, such as one or more of a salt, buffer, oil, organic solvent, enzyme, enzyme byproduct (e.g., glucosyltransferase byproduct such as leucrose, glucose, or gluco-oligosaccharide; e.g., fructose coproduct or unreacted sucrose, both of which are not byproducts per se), surfactant, preservative, personal care product ingredient, household care product ingredient, industrial product ingredient, ingestible product (e.g., food product) ingredient, medical product ingredient, or pharmaceutical product ingredient. Yet, in some aspects, an aqueous composition can consist of, or essentially consist of (e.g., further have one or more salts or buffers), (i) xanthan gum, (ii) insoluble alpha-glucan, and (iii) water; such a composition can optionally be stored and used for later formulation preparation (i.e., addition of one or more other ingredients).

An aqueous composition of the disclosure in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, glucose, leucrose, and/or soluble gluco-oligosaccharides, for example. A composition in some aspects can have one or more salts/buffers (e.g., Na⁺, Cl⁻, NaCl, phosphate, tris, citrate) (e.g., ≤0.1, 0.5, 1.0, 2.0, or 3.0 wt %), and/or a pH of about 4.0, 4.5, 5.0, 5.5, 6.0, 6.5, 7.0, 7.5, 8.0, 8.5, 9.0, 9.5, 10.0, 10.5, 4.0-10.0, 4.0-9.0, 4.0-8.0, 5.0-10.0, 5.0-9.0, 5.0-8.0, 6.0-10.0, 6.0-9.0, or 6.0-8.0, for example.

The temperature of an aqueous composition herein can be about, at least about, or less than about, 0, 5, 10, 15, 20, 25, 30, 35, 37, 40, 42, 45, 50, 55, 60, 65, 70, 75, 80, 85, 90, 95, 100, 105, 110, 115, 120, 125, 130, 10-30, 10-25, 15-50, 15-30, 15-25, 20-40, 20-35, 20-30, 20-25, 25-30, 30-50, 30-45, 30-40, 30-35, 35-40, 35-50, 40-45, 50-60, 110-130, 110-125, 110-120, 115-130, or 115-125° C., for example.

The liquid component of an aqueous composition can be an aqueous fluid such as water or aqueous solution, for instance. The solvent of an aqueous solution typically is water, or can comprise about, or at least about, 10, 20, 30, 40, 50, 60, 70, 80, 90, 95, 98, or 99 wt % water, for example. In some aspects, an aqueous composition herein can comprise, or be in the form of, a dispersion, emulsion, foam, cream, gel, or paste.

An aqueous composition herein can have a viscosity of about, at least about, or less than about, 1, 5, 10, 100, 200, 300, 400, 500, 600, 700, 1000, 2000, 3000, 4000, 5000, 6000, 7000, 8000, 9000, 10000, 15000, 20000, 25000, 1-300, 10-300, 25-300, 50-300, 1-250, 10-250, 25-250, 50-250, 1-200, 10-200, 25-200, 50-200, 1-150, 10-150, 25-150, 50-150, 1-100, 10-100, 25-100, or 50-100 centipoise (cps, cP), for example. Viscosity can be as measured with an aqueous composition herein at any temperature between about 3° C. to about 80° C., for example (e.g., 4-30° C., 15-30° C., 15-25° C.),), or any particular temperature disclosed herein for an aqueous composition. Viscosity typically is as measured at atmospheric pressure (about 760 torr) or a pressure that is ±10% thereof. Viscosity can be measured using a viscometer or rheometer, for example, and can optionally be as measured at a shear rate (rotational shear rate) of about 0.1, 0.3, 0.5, 1.0, 3, 5, 10, 50, 100, 500, 1000, 0.1-500, 0.1-100, 1.0-500, 1.0-1000, or 1.0-100 s⁻¹ (1/s), or about 5, 10, 20, 25, 50, 100, 200, or 250 rpm (revolutions per minute), for example.

Aqueous compositions herein typically have enhanced viscosity (at any given shear rate). For example, an aqueous composition herein can exhibit synergistic enhancement of viscosity, where the viscosity of the aqueous composition exceeds the sum of the viscosities that would have been expected (“expected viscosity”) based on the individual viscosities of counterpart aqueous compositions having only the xanthan gum or the insoluble alpha-glucan (but otherwise having the same other components in the same amounts, and the same conditions). In some aspects, the viscosity of an aqueous composition herein can be about, or at least about, 1.25, 1.5, 2, 2.5, 3, 5, 6, 7, or 8 times the expected viscosity.

In some aspects, insoluble particles of an aqueous composition herein are dispersed through about, or at least about, 30%, 40%, 50%, 60%, 70%, 80%, 85%, 90%, 95%, 98%, 99%, or 100% of the volume of the aqueous composition. In some aspects, such a level of dispersion is contemplated to be for a time (typically beginning from initial preparation of the dispersion) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years. In some aspects, an aqueous composition is a stable emulsion; any of the above dispersal-volume percentages and/or times of such stability can likewise characterize dispersed/emulsified oil droplets. In some aspects, stability can additionally or alternatively characterize an emulsion in which the average emulsion droplet size is relatively small (e.g., about, or less than about, 40, 38, 36, 34, 32, 30, 28, 26, 26-34, 26-32, 26-30, 28-34, 28-32, or 28-30 microns in diameter) and typically uniform in size (e.g., standard deviation of average size about, or less than about, 12, 11, 10, 9, 8, 7, 6, 5, 4, 5-10, 5-8, 6-10, 6-8). A small average droplet size equates to an elevated total droplet surface area. In some aspects, stability can additionally or alternatively characterize an emulsion having an average storage modulus (Avg. G′) (also referred to as elastic modulus) of about, or at least about, 40, 50, 60, 70, 80, 90, 100, 125, 150, 40-150, 40-125, 40-100, 50-150, 50-125, or 50-100 Pascals. The storage modulus of an emulsion herein can be measured according to the below Examples, or as disclosed in Varanasi et al. (2018, Frontiers Chem. 6:1-9, Article 409, incorporated herein by reference), for example. Based on the foregoing dispersion and/or emulsion stability features of an aqueous composition, it is contemplated that an aqueous composition is suitable for use in an application/product in which dispersion or emulsion stabilization improves the performance of the application/product. Examples of such applications/products can be as disclosed herein, such as milk/dairy products (e.g., yogurt, ice cream, cream), mayonnaise, salad dressings, beverages/tonics as carriers for delivering non-polar bioactive ingredients, cosmetic or pharmaceutical lotions/creams/foams/serums, waterborne/latex paints, defoaming formulations, rolling oils for metal working, mining explosives, agrochemical formulations, downhole fluids such as for enhanced oil recovery operations, or pharmaceutical carrier or encapsulation systems.

An aqueous composition herein can, in some aspects, comprise one or more salts such as a sodium salt (e.g., NaCl, Na₂SO₄). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous composition herein at a wt % of about, or at least about, 0.01, 0.025, 0.05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

An aqueous composition herein can optionally contain one or more enzymes (active enzymes). Examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases, nucleases, and amylases. If an enzyme(s) is included, it may be comprised in a composition herein at about 0.0001-0.1 wt % (e.g., 0.01-0.03 wt %) active enzyme (e.g., calculated as pure enzyme protein), for example. In fabric care or automatic dishwashing applications, an enzyme herein (e.g., any of the above such as cellulase, protease, amylase, and/or lipase) can be present in an aqueous composition in which a fabric or dish is treated (e.g., wash liquor, grey water) at a concentration that is minimally about 0.01-0.1 ppm total enzyme protein, or about 0.1-10 ppb total enzyme protein (e.g., less than 1 ppm), to maximally about 100, 200, 500, 1000, 2000, 3000, 4000, or 5000 ppm total enzyme protein, for example.

An aqueous composition herein can optionally contain one or more organic solvents such as a polar organic solvent. A polar organic solvent in some aspects can be protic. Examples of protic polar organic solvents herein include an alcohol (e.g., methanol, ethanol, isopropanol, 1-propanol, tert-butyl alcohol, n-butanol, iso-butanol), methyl formamide and formamide. Additional examples of protic polar organic solvents herein include n-butanol, ethylene glycol, polyethylene glycol, propylene glycol, dipropylene glycol, tripropyleneglycol, polypropylene glycol, 2-methoxyethanol, 1-methoxy-2-propanol, glycerol, 1,2-propanediol, and 1,3-propanetriol. A polar organic solvent in some aspects can be aprotic. Examples of aprotic polar organic solvents herein include acetonitrile, dimethyl sulfoxide, acetone, N,N-dimethylformamide, N,N-dimethylacetamide, tetrahydrofuran, propylene carbonate, and sulfolane. Additional examples of aprotic polar organic solvents herein include hexamethylphosphoramide, dimethylimidazolidinone (1,3-dimethyl-2-imidazolidinone), dioxane, nitromethane, and butanone. In general, ester, ketone and aldehyde solvents having no acidic hydrogen atom are other examples of aprotic polar organic solvents herein. Organic solvent(s) can constitute about, or up to about, 10%, 20%, 30%, 40%, 50%, or 60% by weight or volume of the solvent (balance is water), for example, of an aqueous composition.

An aqueous composition herein can optionally contain one or more preservatives. Examples of preservatives herein include phenoxyethanol, caprylyl glycol, ethylhexylglycerin, benzoate (e.g., sodium benzoate), diazolidinyl urea, iodopropynyl butylcarbamate, 2-methyl-4-isothiazolin-3-one, 5-chloro-2-methyl-4-isothiazolin-3-one, methylcholoroisothiasolinone, methylisothiasolinone, sorbate, benzisothiazolinone, paraben (e.g., methylparaben, ethylparaben, propylparaben, butylparaben, isobutylparaben), nitrate (e.g., sodium nitrate), propionate (e.g., sodium propionate), levulinic acid, anisic acid, formaldehyde, DMDM hydantoin, imadozolidinyl urea, diazolidinyl urea, Germall® II, and Germaben® II.

Xanthan gum and insoluble alpha-glucan in an aqueous composition, or an aqueous composition itself, of the present disclosure typically are biodegradable. Such biodegradability can be, for example, as determined by the Carbon Dioxide Evolution Test Method (OECD Guideline 301B, incorporated herein by reference), to be about, at least about, or at most about, 5%, 10%, 15%, 20%, 25%, 30%, 35%, 40%, 45%, 50%, 55%, 60%, 65%, 70%, 75%, 80%, 85%, 90%, 5-60%, 5-80%, 5-90%, 40-70%, 50-70%, 60-70%, 40-75%, 50-75%, 60-75%, 70-75%, 40-80%, 50-80%, 60-80%, 70-80%, 40-85%, 50-85%, 60-85%, 70-85%, 40-90%, 50-90%, 60-90%, or 70-90%, or any value between 5% and 90%, after 15, 30, 45, 60, 75, or 90 days of testing.

An aqueous composition as presently disclosed can be in the form of a household care product, personal care product, industrial product, ingestible product (e.g., food product), medical product, or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are all incorporated herein by reference. In some aspects, an aqueous composition can comprise at least one component/ingredient of a household care product, personal care product, industrial product, pharmaceutical product, or ingestible product (e.g., food product) as disclosed in any of the foregoing publications and/or as presently disclosed.

An aqueous composition in some aspects is believed to be useful for providing one or more of the following physical properties to a personal care product, pharmaceutical product, household product, industrial product, or ingestible product (e.g., food product): thickening, freeze/thaw stability, lubricity, moisture retention and release, texture, consistency, shape retention, emulsification, binding, suspension, dispersion, gelation, reduced mineral hardness, for example.

Personal care products herein are not particularly limited and include, for example, skin care compositions, cosmetic compositions, antifungal compositions, and antibacterial compositions. Personal care products herein may be in the form of, for example, lotions, creams, foams, pastes, balms, ointments, pomades, gels, liquids, serums, combinations of these and the like. The personal care products disclosed herein can include at least one active ingredient, if desired. An active ingredient is generally recognized as an ingredient that causes an intended pharmacological effect.

A personal care product in some aspects can be a skin care product. A skin care product can be used on, and/or be designed for, general body application or targeted application (e.g., to hands or feet), for example. A skin care product in some aspects can be used on hair and/or nails (or exclusively for nails) in some aspects. In some aspects, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil. A skin care product can be an ointment, lotion, or sanitizer (e.g., hand sanitizer) in some aspects. A skin care product/formulation that can be adapted to be an aqueous composition herein can be as disclosed in, for example, US20100189669, US20200093799, US20080014162, US20050002889, US20020039565, US20080213323, US20040022822, US20070166249, US20080152606, US20080008668, US20140256830, US20030206932, US20030114323, US20110152335, US20150202139, US20040180026, U.S. Pat. Nos. 4,595,586, 4,268,526, 4,272,519, 4,285,967, 4,368,189, 4,372,944, 4,699,780, 4,816,271, 4,839,164, 4,464,362, 5,552,135, 5,693,255, 5,976,555, 5,607,921, 5,618,523, 5,798,108, 5,356,627, 5,811,083, 5,939,085, 6,280,714, 8,465,973, 9,867,774, U.S. Ser. No. 11/110,049, U.S. Ser. No. 10/546,658, U.S. Ser. No. 11/033,480, EP0321929, or WO2013092872, all of which are incorporated herein by reference. A skin care product can comprise one or more ingredients/additives as disclosed in any of the foregoing references, for example.

A personal care product herein can also be in the form of makeup, lipstick, mascara, rouge, foundation, blush, eyeliner, lip liner, lip gloss, other cosmetics, sunscreen, sun block, nail polish, nail conditioner, bath gel, shower gel, body wash, face wash, lip balm, skin conditioner, cream, foam, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, serum (skin serum), rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse, hair spray, and styling gel. A hair care product can be in the form of a liquid, paste, gel, cream, foam, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

An personal care product in some aspects can be a hair care composition such as a hair styling or hair setting composition (e.g., hair gel or lotion, hair mousse/foam, hair serum) (e.g., foam, creme, paste, non-runny gel, mousse, pomade, lacquer, hair wax). A hair styling/setting composition/formulation that can be adapted to be an aqueous composition herein can be as disclosed in, for example, US20090074697, WO1999048462, US20130068849, JPH0454116A, U.S. Pat. No. 5,304,368, AU667246B2, U.S. Pat. Nos. 5,413,775, 5,441,728, 5,939,058, JP2001302458A, U.S. Pat. No. 6,346,234, US20020085988, U.S. Pat. No. 7,169,380, US20090060858, US20090326151, US20160008257, WO2020164769, or US20110217256, all of which are incorporated herein by reference. A hair care composition such as a hair styling/setting composition can comprise one or more ingredients/additives as disclosed in any of the foregoing references, and/or one or more of a fragrance/perfume, aroma therapy essence, herb, infusion, antimicrobial, stimulant (e.g., caffeine), essential oil, hair coloring, dying or tinting agent, anti-gray agent, anti-foam agent, sunscreen/UV-blocker (e.g., benzophenone-4), vitamin, antioxidant, surfactant or other wetting agent, mica, silica, metal flakes or other glitter-effect material, conditioning agent (e.g., a volatile or non-volatile silicone fluid), anti-static agent, opacifier, detackifying agent, penetrant, preservative (e.g., phenoxyethanol, ethylhexylglycerin, benzoate, diazolidinyl urea, iodopropynyl butylcarbamate), emollient (e.g., panthenol, isopropyl myristate), rheology-modifying or thickening polymer (e.g., acrylates/methacrylamide copolymer, polyacrylic acid [e.g., CARBOMER]), emulsified oil phase, petrolatum, fatty alcohols, diols and polyols, emulsifier (e.g., PEG-40 hydrogenated castor oil, Oleth-20), humectant (e.g., glycerin, caprylyl glycol), silicone derivative, protein, amino acid (e.g., isoleucine), conditioner, chelant (e.g., EDTA), solvent (e.g., see below), monosaccharide (e.g., dextrose), disaccharide, oligosaccharide, pH-stabilizing compound (e.g., aminomethyl propanol), film former (e.g., acrylates/hydroxyester acrylate copolymer, polyvinylpyrrolidone/vinyl acetate copolymer, triethyl acetate), and/or any other suitable material herein. Optional hair fixing/styling agents herein include PVP (polyvinylpyrrolidone), octylacrylamide/acrylates/butylaminoethyl methacrylate copolymer, vinyl caprolactam/PVP/dimethylaminoethyl methacrylate copolymer, AMPHOMER, or any film former such as listed above.

A hair styling/setting composition can comprise a solvent comprising water and optionally a water-miscible (typically polar) organic compound (e.g., liquid or gas) such as an alcohol (e.g., ethanol, propanol, isopropanol, n-butanol, iso-butanol, tert-butanol), an alkylene glycol alkyl ether, and/or a monoalkyl or dialkyl ether (e.g., dimethyl ether), for example. If an organic compound is included, it can constitute about 10%, 20%, 30%, 40%, 50%, or 60% by weight or volume of the solvent (balance is water), for example. The amount of solvent in a hair styling/setting composition herein can be about 50-90, 60-90, 70-90, 80-90, 50-95, 60-95, 70-95, 80-95, or 90-95 wt %, for example.

Various examples of personal care formulations comprising at least xanthan gum and insoluble alpha-glucan as presently disclosed are disclosed below (1-3, content percentages in wt %).
(1) A skin lotion composition according to Tables 1-2 herein comprising xanthan gum and insoluble alpha-glucan, but where the amount of each ingredient (xanthan gum, insoluble alpha-glucan, glycerin, sodium PEG-7 olive oil carboxylate [e.g., Olivem® 400], phenoxyethanol [e.g., Neolone™ PH100], isohexadecane, isopropyl isostearate [e.g., CRODAMOL™ IPIS-LQ-(MV)], dimethicone [e.g., XIAMETER™ PMX-200 Silicone Fluid 100 cST], glyceryl stearate-PEG-100 stearate [e.g., Alkamuls® GS 100], cetearyl glucoside-cetearyl alcohol [e.g., Montanov™ 68 MB], stearic acid, alkaline hydroxide [e.g., NaOH], water) is within 5%, 10%, 15%, or 20% of the amount listed in Tables 1-2.
(2) A skin lotion composition comprising: 1-5% glycerin, 1-5% glycol stearate, 1-5% stearic acid, 1-5% mineral oil, 0.5-1% acetylated lanolin (Lipolan® 98), 0.1-0.5% cetyl alcohol, 0.2-1% triethanolamine, 0.1-1% Germaben® II preservative, 0.5% xanthan gum, insoluble alpha-glucan (0.3%, 0.5%, 0.75%, 1.0%, or 1.5%), and the balance being water.
(3) A serum composition according to Tables 3-4 herein comprising xanthan gum and insoluble alpha-glucan, but where the amount of each ingredient (xanthan gum, insoluble alpha-glucan, hyaluronic acid, propylene glycol, glycerin, disodium EDTA, betaine [e.g., Genencare® BA], PEG-40 hydrogenated castor oil [e.g., Cremophor® RH 40], dimethicone [e.g., XIAMETER™ PMX-200 Silicone Fluid 100 cST], alkaline hydroxide [e.g., NaOH], water) is within 5%, 10%, 15%, or 20% of the amount listed in Tables 3-4.

A pharmaceutical product herein can be in the form of an emulsion, liquid, elixir, gel, suspension, solution, cream, foam, serum, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. A composition herein can also be used in capsules, encapsulants, tablets, tablet coatings, and as an excipients for medicaments and drugs.

A household care and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; air fresheners; polymer emulsions; latex; gels such as water-based gels; surfactant solutions; paints such as water-based paints; protective coatings; adhesives; sealants and caulks; inks such as water-based ink; metal-working fluids; films or coatings; or emulsion-based metal cleaning fluids used in electroplating, phosphatizing, galvanizing and/or general metal cleaning operations, for example. In some aspects, a composition herein is comprised in a fluid as a viscosity modifier and/or friction reducer; such uses include downhole operations/fluids (e.g., in hydraulic fracturing and enhanced oil recovery).

Examples of ingestible products herein include a food, beverage, animal feed, an animal health and/or nutrition product, and/or pharmaceutical product. The intended use of a composition as presently disclosed in an ingestible product can be to provide texture, add volume, and/or thicken, for example.

Further examples of using a composition of the present disclosure for ingestible products include use as: a bulking, binding and/or coating ingredient; a carrier for coloring agents, flavors/fragrances, and/or high intensity sweeteners; a spray drying adjunct; a bulking, bodying, dispersing and/or emulsification agent; and an ingredient for promoting moisture retention (humectant). Illustrative examples of products that can be prepared having a composition herein include food products, beverage products, pharmaceutical products, nutritional products, and sports products. Examples of beverage products herein include concentrated beverage mixes, carbonated beverages, non-carbonated beverages, fruit-flavored beverages, fruit juices, teas, coffee, milk nectars, powdered drinks, liquid concentrates, milk drinks, ready-to-drink (RTD) products, smoothies, alcoholic beverages, flavored waters and combinations thereof. Examples of food products herein include baked goods (e.g., breads), confectioneries, frozen dairy products, meats, artificial/synthetic/cultured meat, cereal products (e.g., breakfast cereals), dairy products (e.g., yogurt), condiments (e.g., mustard, ketchup, mayonnaise), snack bars, soups, dressings, mixes, prepared foods, baby foods, diet preparations, peanut butter, syrups, sweeteners, food coatings, pet food, animal feed, animal health and nutrition products, dried fruit, sauces, gravies, jams/jellies, dessert products, spreads, batters, breadings, spice mixes, frostings and the like. In some aspects, a composition herein can provide or enhance the foaming of beverages such as dairy beverages, non-dairy alternative beverages (e.g., “vegan” milk such as soy milk, almond milk, or coconut milk), dairy creamers, and/or non-dairy creamers (e.g., for a hot beverage such as coffee [e.g., cappuccino], tea [e.g., chai tea]).

Some aspects herein regard a method/process of producing an aqueous composition as presently disclosed. Such a method can comprise: blending together at least water, xanthan gum, and insoluble alpha-glucan as presently disclosed. One or more additional ingredients, such as any disclosed herein, can also be blended with the water, xanthan gum and insoluble alpha-glucan in some aspects. Such a method can optionally be characterized as a method of blending or formulating. An aqueous composition of the present disclosure can be made by such a method, for example.

The order of adding each ingredient for blending in this method can vary. Merely for example, xanthan gum can first be dissolved in water, followed by dispersing the insoluble alpha-glucan therein, and then adding any additional ingredient(s). As another example, insoluble alpha-glucan can first be dispersed in water, followed by dissolving the xanthan gum into the preparation and then blending in other ingredients. In some aspects, a method of blending (e.g., of water, xanthan gum and insoluble alpha-glucan, or these plus one or more additional ingredients) can be performed as disclosed in the below Examples, but with conditions/parameters (e.g., temperatures, pH values, mixing speeds, time periods, and/or concentrations) that are within 5%, 10%, 15%, and/or 20% of the disclosed conditions/parameters.

Non-limiting examples of compositions and methods disclosed herein include:
1. An aqueous composition (product) comprising xanthan gum and insoluble alpha-glucan, wherein: (i) at least about 50% of the glycosidic linkages of the insoluble alpha-glucan are alpha-1,3 glycosidic linkages, (ii) the weight-average degree of polymerization (DPw) of the insoluble alpha-glucan is at least 10, and (iii) the insoluble alpha-glucan is in the form of particles having a degree of crystallinity of at least about 0.65.
2. The aqueous composition of embodiment 1, wherein at least about 90% of the glycosidic linkages of the insoluble alpha-glucan are alpha-1,3 glycosidic linkages.
3. The aqueous composition of embodiment 1 or 2, wherein the DPw of the insoluble alpha-glucan is about 15 to 100.
4. The aqueous composition of embodiment 1 or 2, wherein the DPw of the insoluble alpha-glucan is about 35 to about 100.
5. The aqueous composition of embodiment 1 or 2, wherein the DPw of the insoluble alpha-glucan is about 35 to about 60.
6. The aqueous composition of embodiment 1, 2, 3, 4, or 5, wherein the insoluble alpha-glucan particles have a degree of crystallinity of at least about 0.7.
7. The aqueous composition of embodiment 1, 2, 3, 4, 5, or 6, comprising about 0.05% to 5% by weight of the insoluble alpha-glucan.
8. The aqueous composition of embodiment 7, comprising about 0.05% to 1% by weight of the insoluble alpha-glucan.
9. The aqueous composition of embodiment 8, comprising about 0.1% to 1% by weight of the insoluble alpha-glucan.
10. The aqueous composition of embodiment 1, 2, 3, 4, 5, 6, 7, 8, or 9, comprising about 0.05% to 2% by weight of the xanthan gum.
11. The aqueous composition of embodiment 10, comprising about 0.05% to 1% by weight of the xanthan gum.
12. The aqueous composition of embodiment 1, 2, 3, 4, 5, 6, 7, 8, 9, 10, or 11, comprising at least about 50% by weight water.
13. The aqueous composition of embodiment 12, comprising at least about 70% by weight water.
14. The aqueous composition of embodiment 1, 2, 3, 4, 5, 6, 7, 8, 9, 10, 11, 12, or 13, wherein the composition is a dispersion.
15. The aqueous composition of embodiment 1, 2, 3, 4, 5, 6, 7, 8, 9, 10, 11, 12, or 13, wherein the composition is an emulsion.
16. The aqueous composition of embodiment 1, 2, 3, 4, 5, 6, 7, 8, 9, 10, 11, 12, 13, 14, or 15, wherein the composition is a household care product, personal care product, industrial product, ingestible product (e.g., food product), or pharmaceutical product.
17. The aqueous composition of embodiment 1, 2, 3, 4, 5, 6, 7, 8, 9, 10, 11, 12, 13, 14, 15, or 16, wherein the composition is a lotion or serum, which typically is of a personal care product or pharmaceutical product.
18. The aqueous composition of embodiment 17, wherein the lotion or serum is a skin care product.
19. The aqueous composition of embodiment 1, 2, 3, 4, 5, 6, 7, 8, 9, 10, 11, 12, 13, 14, 15, 16, 17, or 18, wherein the composition has enhanced stability (e.g., stable dispersion or stable emulsion).
20. The aqueous composition of embodiment 1, 2, 3, 4, 5, 6, 7, 8, 9, 10, 11, 12, 13, 14, 15, 16, 17, 18, or 19, wherein the composition has enhanced viscosity.
21. A method of producing an aqueous composition according to embodiment 1, 2, 3, 4, 5, 6, 7, 8, 9, 10, 11, 12, 13, 14, 15, 16, 17, 18, 19 or 20, the method comprising: blending together at least water, the xanthan gum, and the insoluble alpha-glucan.
22. The aqueous composition of embodiment 1, 2, 3, 4, 5, 6, 7, 8, 9, 10, 11, 12, 13, 14, 15, 16, 17, 18, 19 or 20, but wherein guar gum is in addition to the xanthan gum, or in place of the xanthan gum (e.g., the guar gum is provided at any concentration as disclosed herein for xanthan gum).

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Materials/Methods

### Representative Preparation of Highly Crystalline Insoluble Alpha-1,3-Glucan (Microcrystalline Alpha-1,3-Glucan [MCG])

In general, highly crystalline alpha-1,3-glucan can be prepared as described below, and/or by following methodology disclosed in U.S. Patent Appl. Publ. No. 2021/0130504, which is incorporated herein by reference.

Insoluble alpha-1,3-glucan was first prepared by enzymatic synthesis in a manner similar to what is described in U.S. Patent Appl. Publ. Nos. 2018/0340199 and 2019/0078063, which are both incorporated herein by reference. In general, a glucan synthesis reaction was performed comprising water, sucrose, buffer, filtrate from an earlier glucan synthesis reaction (contains, e.g., gluco-oligosaccharide byproducts of the earlier glucan synthesis reaction), and an amino acid-modified, high product-yielding glucosyltransferase enzyme. Following the reaction, the alpha-1,3-glucan product (insoluble, ˜100% alpha-1,3 linkages, DPw of about 800) was filtered and washed to remove most fructose and other residual soluble sugars (e.g., glucose, sucrose, leucrose, DP2-DP8 gluco-oligosaccharides). Samples of the washed product were then either collected into wet cakes (never-dried) of about 20-40 wt % solids or dried in a rotary dryer to powders of about 88-95 wt % solids.

Samples of both never-dried and dried insoluble alpha-1,3-glucan were then subjected to hydrochloric acid hydrolysis procedures at a pH of almost 0 at 80° C. to produce reduced molecular weight insoluble alpha-1,3-glucan. Each hydrolysis reaction as initiated contained 8 wt % alpha-1,3-glucan. Procedures disclosed in U.S. Patent Appl. Publ. No. 2013/0244287 (incorporated herein by reference), which describes mineral acid hydrolysis of insoluble alpha-1,3-glucan to soluble alpha-1,3-glucan, can be applied with appropriate modification to hydrolyze alpha-1,3-glucan to a lower molecular weight, but insoluble, form. Hydrolysis reactions were allowed to proceed for 1 hour, 8 hours, 1 day, or 3 days before being neutralized. Each hydrolyzed, insoluble alpha-1,3-glucan product was then analyzed for molecular weight. Insoluble alpha-1,3-glucan with a weight-average degree of polymerization (DPw) of roughly 40-60 was produced after one day of hydrolysis of either never-dried or dried insoluble alpha-1,3-glucan. This molecular weight was stable, remaining at a similar DPw for the duration of hydrolysis under the very low pH conditions. In a separate hydrolysis, insoluble alpha-1,3-glucan with a DPw of about 39 was produced.

Crystallinity (or crystallinity index [CI]) of the alpha-1,3-glucan samples was measured by wide-angle X-ray scattering (WAXS) as follows. Glucan powder samples were dried for a minimum of two hours or overnight (but sometimes over the weekend) in a vacuum oven set at 60° C. Immediately before starting the diffraction scan, each sample was removed from the oven and transferred into a stainless steel holder with a well of about 1.5 cm wide by 4 cm long by 4 mm deep. The well was open at the side such that powder could be poured in through the side, with a glass plate clipped onto the top of the holder. The powder was packed down several times throughout the filling process by hitting the opposite side of the holder against the table repeatedly. Finally, the holder was turned right-side-up, the glass plate was removed, and the holder was loaded into a diffractometer. The time from the opening of the oven to the start of the scan was five minutes or less. An X'PERT MPD POWDER diffractometer (PANalytical B.V., The Netherlands) in reflection mode was used to measure the X-ray diffraction pattern of each powder sample. The X-ray source was a Cu X-ray tube line source with an optical focusing mirror and a 1/16° narrowing slit. X-rays were detected with a 1-D detector and an anti-scatter slit set at ⅛°. Data were collected in the range of 4 to 60 degrees of two-theta at 0.1 degrees per step. The scan took about 46 minutes in total. The resulting X-ray pattern was then analyzed by subtracting a linear baseline from 7.2 to 30.5 degrees, subtracting the XRD pattern of a known amorphous alpha-1,3-glucan sample that had been scaled to fit the current data, and then fitting the remaining crystal peaks in that range with a series of Gaussian curves corresponding to known dehydrated alpha-1,3-glucan crystal reflections. The area corresponding to the crystal peaks was then divided by the total area under the baseline-subtracted curve to yield a crystallinity index.

The crystallinity of the alpha-1,3-glucan samples prepared above by hydrolysis was compared to the crystallinity of enzymatically polymerized alpha-1,3-glucan that was not subjected to hydrolysis. The hydrolyzed alpha-1,3-glucan had substantially greater crystallinity (over 0.65) compared to non-hydrolyzed alpha-1,3-glucan. In particular, hydrolyzed alpha-1,3-glucan with a DPw of 50 (made by acid-hydrolyzing, as above, wet cake for 48 hours at 40° C.) had a crystallinity of about 0.76. A sample of hydrolyzed alpha-1,3-glucan with a DPw of 94 (made by acid-hydrolyzing, as above, wet cake for 1 hour at 40° C.) had a crystallinity of about 0.69. However, samples of non-hydrolyzed alpha-1,3-glucan (˜100% alpha-1,3 linkages) produced enzymatically and having DPw values ranging from ˜230 to ˜830 had lower crystallinities (the molecular weight of alpha-1,3-glucan as produced enzymatically can be modulated to be within the range of DPw 230-830 using a technique as described in, for example, U.S. Patent Appl. Publ. No. 2015/0064748, which is incorporated herein by reference).

Using electron microscopy, the microstructure of hydrolyzed alpha-1,3-glucan (DPw 50, 0.76 Cl, 1.2 PDI) was compared to that of non-hydrolyzed alpha-1,3-glucan (DPw ˜800) (as produced above). The glucan samples were imaged by dry-cast electron microscopy using phosphotungstate as a contrast agent, as follows. Slurries of DPw 50 and DPw ˜800 alpha-1,3-glucan were purified by multiple rounds of centrifugation and redispersion into DI water. The final purified glucan samples were diluted 100-fold and then sonicated for 3 minutes. Once sonication was completed, supernatant from each preparation was isolated to prepare a dry-cast transmission electron microscopy (TEM) sample on a copper mesh TEM grid. Phosphotungstic acid was then used for negative contrast staining, after which TEM imaging was performed. The captured TEM images usually were from sections located at the edge of a larger thick sample deposited on the TEM grid. The hydrolyzed alpha-1,3-glucan (DPw 50) exhibited two-dimensional structures (>about 90 wt % of material that was not aggregated was in the form of plates), whereas the non-hydrolyzed alpha-1,3-glucan (DPw ˜800) exhibited larger, three-dimensional fibrillar structures. TEM imaging of non-hydrolyzed alpha-1,3-glucan (˜100% alpha-1,3 linkages) produced enzymatically and having a DPw of about 260 showed a microstructure very similar to that of non-hydrolyzed alpha-1,3-glucan (DPw ˜800).

### General Procedure for Preparing Aqueous Compositions Comprising Xanthan Gum and Microcrystalline Alpha-1,3-Glucan

Xanthan gum (Keltrol® CG-RD, CP Kelco) was added to water under agitation. After the xanthan gum was fully dissolved, insoluble microcrystalline alpha-1,3-glucan (MCG) (DPw 40-50, ˜100% alpha-1,3 linkages, 0.76 Cl [crystallinity index]) (supplied as an 8-12 wt % dispersion) was added according to the desired dosage. A diluted sodium hydroxide solution was used to adjust the pH of each preparation to 5.8.

### General Procedure for Preparing Personal Care Lotion

1. Phase A (Table 1) was prepared by (a) adding xanthan gum to water under overhead stirring, followed by heating to ˜75° C. until there were no visible agglomerates (the same procedure was used if using Acrylates/C10-30 Alkyl Acrylate Crosspolymer [Carbopol® Ultrez 10 or 20]); (b) adding the appropriate amount of MCG for each targeted concentration (below); and (c) adding all other ingredients of Phase A.
2. Phase B (Table 1) was prepared by adding all components in a bottle and heating to −75° C. until everything dissolved to a homogeneous solution.
3. Transferred Phase B to Phase A under overhead mixing.
4. Emulsified the preparation with a rotor-stator high shear mixer (Silverson L5) at 6000-7000 rpm for 2 minutes.
5. Cooled the preparation to ambient temperature under overhead mixing.
6. Adjusted pH of the preparation with Phase C (Table 1).

TABLE 1

| Phase | Ingredient | wt % |
|---|---|---|
| A | Deionized Water | q.s. to 100 |
| | Thickener(s) (Table 2) | as defined |
| | | (Table 2) |
| | Glycerin | 5 |
| | Sodium PEG-7 Olive Oil Carboxylate | 0.5 |
| | (Olivem ® 400) | |
| | Phenoxyethanol | 0.5 |
| | (Neolone ™ PH100) | |
| B | Isohexadecane | 5 |
| | Isopropyl Isostearate | 5 |
| | (CRODAMOL ™ IPIS-LQ-(MV)) | |
| | Dimethicone | 2 |
| | (XIAMETER ™ PMX-200 Silicone | |
| | Fluid 100 cST) | |
| | Glyceryl Stearate, PEG-100 Stearate | 0.5 |
| | (Alkamuls ® GS 100) | |
| | Cetearyl Glucoside, Cetearyl Alcohol | 0.5 |
| | (Montanov ™ 68 MB) | |
| | Stearic Acid | 0.5 |
| C | Sodium Hydroxide | q.s. to pH |
| | | 5.8 |

Lotion samples were prepared following the above protocol with various combinations of thickeners as listed in Table 2.

TABLE 2

Thickener

| | | Xanthan | Carbopol | Carbopol |
|---|---|---|---|---|
| Sample ID | MCG | Gum | Ultrez 10 | Ultrez 20 |
| Example 1 | 1.5 | 0.5 | — | — |
| Example 2 | 1.0 | 0.5 | — | — |
| Example 3 | 0.75 | 0.5 | — | — |
| Example 4 | 0.5 | 0.5 | — | — |
| Example 5 | 0.3 | 0.5 | — | — |
| Control 1 | — | 0.5 | — | — |
| Control 2 | 1.0 | — | — | — |
| Control 3 | 0.3 | — | — | — |
| Benchmark 1 | — | — | 0.1 | — |
| Benchmark 2 | — | — | — | 0.1 |

Note:
All listed values are in wt %.

### General Procedure for Preparing Personal Care Serum

1. Added xanthan gum to water under overhead stirring until there were no visible solids.
2. Added ingredients 3-9 (Table 3) in order. For Example 6, first added MCG and then ingredients 3-9.
3. Mixed the preparation with a rotor-stator high shear mixer (Silverson L5) at 6000-7000 rpm for 2 minutes.
4. Adjusted the pH of the preparation with diluted sodium hydroxide solution (ingredient 10, Table 3).

TABLE 3

| No. | Ingredient | wt % |
|---|---|---|
| 1 | Deionized Water | q.s. to 100 |
| 2 | Thickener(s) | as defined (Table 4) |
| 3 | Hyaluronic Acid | 0.15 |
| 4 | Propylene Glycol | 4 |
| 5 | Glycerin | 5 |
| 6 | Disodium EDTA | 0.05 |
| 7 | Water, Betaine | 3 |
| | (Genencare ® BA) | |
| 8 | PEG-40 Hydrogenated Castor Oil | 3 |
| | (Cremophor ® RH 40) | |
| 9 | Dimethicone | 2 |
| | (XIAMETER ™ PMX-200 Silicone | |
| | Fluid 100 cST) | |
| 10 | Sodium Hydroxide | q.s. to pH 5.8 |

Serum samples were prepared following the above protocol with various combinations of thickeners as listed in Table 4.

TABLE 4

Thickener

| | | Xanthan |
|---|---|---|
| Sample ID | MCG | Gum |
| Example 6 | 0.4 | 0.2 |
| Control 4 | | 0.2 |

Note:
All listed values are in wt %.

### Viscosity Measurement

Sample viscosities were evaluated using a TA Discovery Hybrid Rheometer with a DIN concentric cylinder. Each test was conducted with a flow ramp of 0.1 to 1000 s⁻¹ at 25° C.

### Emulsion Stability Evaluation

Lotion and serum samples were loaded into LUMiSizer® cells and subjected to centrifugation at 4000 rpm at 40° C. for 4 hours. The instability index for each sample was calculated using the SEPView® software of LUMiSizer®. The instability index range was 0 to 1, where a higher value indicated that a sample was more unstable.

### Results

The viscosities in water at various shear rates of 3 wt % MCG, 0.5 wt % xanthan gum and their corresponding combination (combo) are listed in Table 5A. The synergy index for each sample/shear rate was also calculated (Table 5A). The synergy index was defined as

$$\frac{{η}_{combo}}{{η}_{MCG} + {η}_{xanthan}} ,$$

where η is the viscosity of a sample at a particular shear rate; the sample viscosities entered into the above formula were all measured at the same shear rate. Notably, the synergy indices for the MCG/xanthan gum combinations were all greater than 1 as measured with a shear rate up to 100 s⁻¹. Higher synergy indices were observed as the shear rate was lowered.

TABLE 5A
Sample Viscosity at Various Shear Rates (3 wt % MCG)

| | Shear Rate | | | | | | | | | |
|---|---|---|---|---|---|---|---|---|---|---|
| Sample | 0.3 s⁻¹ | | 1 s⁻¹ | | 3 s⁻¹ | | 10 s⁻¹ | | 100 s⁻¹ | |
| 3 wt % | 1014 | cP | 340 | cP | 150 | cP | 51 | cP | 13.8 | cP |
| MCG | | | | | | | | | | |
| 0.5 wt % | 1396 | cP | 881 | cP | 600 | cP | 278 | cP | 70.4 | cP |
| Xanthan | | | | | | | | | | |
| Gum | | | | | | | | | | |
| Combo | 10422 | cP | 3743 | cP | 1778 | cP | 573 | cP | 90.8 | cP |

| | | | | | |
|---|---|---|---|---|---|
| Synergy | 4.3 | 3.1 | 2.4 | 1.7 | 1.1 |
| Index | | | | | |

Furthermore, lowering the content of MCG to 1 wt % (Table 5B) or 0.3 wt % (Table 5C), as in combination (combo) with 0.5 wt % xanthan gum, resulted in even higher synergy indices for several of the combinations at different shear rates.

TABLE 5B
Sample Viscosity at Various Shear Rates (1 wt % MCG)

| | Shear Rate | | | | | | | | | |
|---|---|---|---|---|---|---|---|---|---|---|
| Sample | 0.3 s⁻¹ | | 1 s⁻¹ | | 3 s⁻¹ | | 10 s⁻¹ | | 100 s⁻¹ | |
| 1 wt % | 46 | cP | 22 | cP | 14 | cP | 10 | cP | 7 | cP |
| MCG | | | | | | | | | | |
| 0.5 wt % | 1396 | cP | 881 | cP | 600 | cP | 278 | cP | 70.4 | cP |
| Xanthan | | | | | | | | | | |
| Gum | | | | | | | | | | |
| Combo | 10130 | cP | 3363 | cP | 1656 | cP | 545 | cP | 82.5 | cP |

| | | | | | |
|---|---|---|---|---|---|
| Synergy | 7.0 | 3.7 | 2.7 | 1.9 | 1.1 |
| Index | | | | | |

TABLE 5C
Sample Viscosity at Various Shear Rates (0.3 wt % MCG)

| | Shear Rate | | | | | | | | | |
|---|---|---|---|---|---|---|---|---|---|---|
| Sample | 0.3 s⁻¹ | | 1 s⁻¹ | | 3 s⁻¹ | | 10 s⁻¹ | | 100 s⁻¹ | |
| 0.3 wt % | 18 | cP | 9 | cP | 8 | cP | 7 | cP | 7 | cP |
| MCG | | | | | | | | | | |
| 0.5 wt % | 1396 | cP | 881 | cP | 600 | cP | 278 | cP | 70.4 | cP |
| Xanthan | | | | | | | | | | |
| Gum | | | | | | | | | | |
| Combo | 10956 | cP | 3110 | cP | 1399 | cP | 420 | cP | 62.1 | cP |

| | | | | | |
|---|---|---|---|---|---|
| Synergy | 7.7 | 3.5 | 2.3 | 1.5 | 0.8 |
| Index | | | | | |

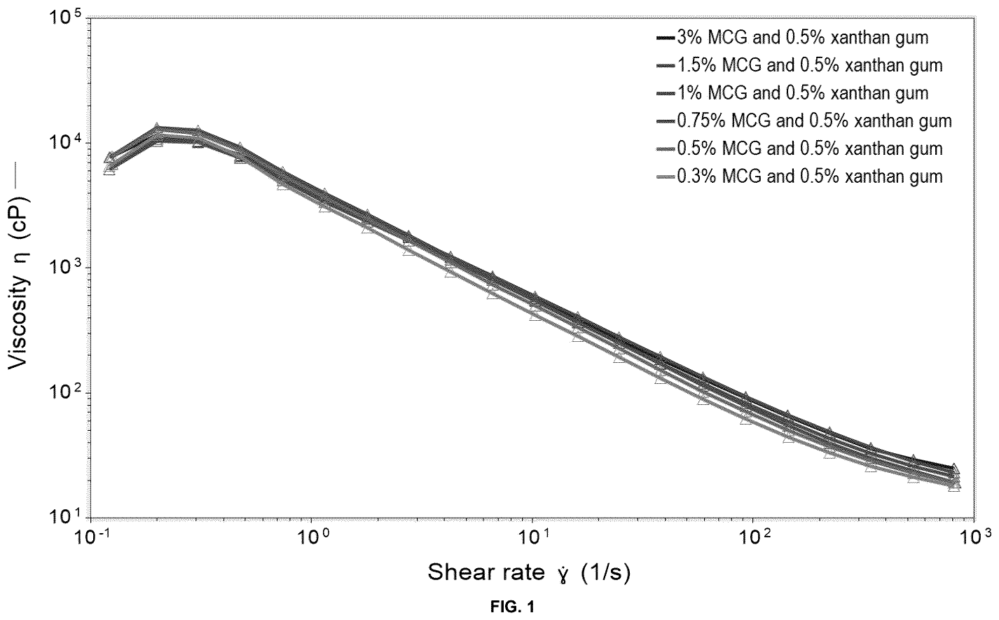

Various additional combinations of MCG and xanthan gum in water were prepared and analyzed for viscosity across of range of shear rates. In particular, six different samples were made, each having 0.5 wt % xanthan gum, but with 3.0, 1.5, 1.0, 0.75, 0.5, or 0.3 wt % MCG, and viscosities were measured at various shear rates between about 0.1 and 1000 s⁻¹. Notably, all the samples displayed similar viscosity levels at each selected shear rate, particularly at shear rates of less than 10 s⁻¹ (FIG. 1); these viscosity data are also provided below in table format (Table A).

TABLE A

| | Viscosity η (cP) | | | | | |
|---|---|---|---|---|---|---|
| | 3.0% | 1.5% | 1% | 0.75% | 0.5% | 0.3% |
| Shear | MCG + | MCG + | MCG + | MCG + | MCG + | MCG + |
| rate | 0.5% | 0.5% | 0.5% | 0.5% | 0.5% | 0.5% |
| (1/s) | xanthan | xanthan | xanthan | xanthan | xanthan | xanthan |
| 0.1 | 7743 | 6840 | 6199 | 7699 | 7806 | 6581 |
| 0.2 | 11588 | 10800 | 10477 | 13406 | 12895 | 11583 |
| 0.3 | 10422 | 10327 | 10130 | 12649 | 11970 | 10956 |
| 0.5 | 8000 | 7881 | 7632 | 9352 | 8737 | 7779 |
| 0.7 | 5566 | 5315 | 5035 | 5917 | 5491 | 4664 |
| 1.1 | 3743 | 3581 | 3364 | 3959 | 3668 | 3110 |
| 1.8 | 2555 | 2503 | 2343 | 2699 | 2460 | 2093 |
| 2.8 | 1779 | 1776 | 1656 | 1831 | 1653 | 1399 |
| 4.3 | 1232 | 1252 | 1163 | 1235 | 1107 | 935 |
| 6.6 | 843 | 868 | 803 | 823 | 739 | 623 |
| 10.3 | 573 | 597 | 545 | 551 | 498 | 420 |
| 16.0 | 389 | 408 | 367 | 371 | 335 | 285 |
| 24.7 | 269 | 281 | 250 | 252 | 226 | 194 |
| 38.1 | 186 | 195 | 172 | 170 | 152 | 131 |
| 59.3 | 129 | 135 | 118 | 115 | 103 | 89 |
| 92.4 | 91 | 94 | 82 | 79 | 71 | 62 |
| 143.8 | 65 | 67 | 59 | 55 | 50 | 44 |
| 221.5 | 48 | 49 | 43 | 40 | 37 | 33 |
| 341.6 | 36 | 37 | 33 | 30 | 28 | 26 |
| 531.9 | 29 | 28 | 26 | 24 | 23 | 21 |
| 810.4 | 25 | 23 | 21 | 19 | 19 | 18 |

The viscosities of lotions comprising MCG, xanthan gum, or their corresponding combination were measured at various shear rates (Table 6). Notably, a synergy effect was observed on viscosity in lotion having both thickeners (Table 6, Example 2).

TABLE 6
Lotion Sample Viscosity at Various Shear Rates

| | Shear Rate | | | | | | | | | |
|---|---|---|---|---|---|---|---|---|---|---|
| Sample | 0.3 s⁻¹ | | 1 s⁻¹ | | 3 s⁻¹ | | 10 s⁻¹ | | 100 s⁻¹ | |
| Control 2 | 2434 | cP | 1034 | cP | 589 | cP | 261 | cP | 78 | cP |
| (1 wt % | | | | | | | | | | |
| MCG) | | | | | | | | | | |
| Control 1 | 14378 | cP | 4832 | cP | 2301 | cP | 765 | cP | 143 | cP |
| (0.5% | | | | | | | | | | |
| Xanthan | | | | | | | | | | |
| Gum) | | | | | | | | | | |
| Example 2 | 22612 | cP | 8210 | cP | 3987 | cP | 1358 | cP | 254 | cP |
| (1 wt % | | | | | | | | | | |
| MCG, 0.5 | | | | | | | | | | |
| wt % | | | | | | | | | | |
| Xanthan | | | | | | | | | | |
| Gum) | | | | | | | | | | |
| Synergy | 1.3 | | 1.4 | | 1.4 | | 1.3 | | 1.1 | |
| Index | | | | | | | | | | |

Lotions comprising MCG, xanthan gum, or their corresponding combination were tested for emulsion stability, along with lotions made having incumbent thickeners (Carbopol® Ultrez 10 or 20). As shown in Table 7 (Example 2), lotion with both MCG and xanthan gum exhibited superior emulsion stability as compared to the emulsion stability of lotions having incumbent thickeners.

TABLE 7
Lotion Emulsion Stability

| | Instability |
|---|---|
| Sample | Index |
| Control 2 | 0.766 |
| (1 wt % MCG) | |
| Control 1 | 0.005 |
| (0.5% Xanthan Gum) | |
| Example 2 | 0.002 |
| (1.0 wt % MCG, 0.5 wt % Xanthan Gum) | |
| Benchmark 1 | 0.415 |
| Benchmark 2 | 0.156 |

All lotion samples of Examples 1-5 displayed excellent emulsion stability without visible phase separation (Table 8).

TABLE 8
Lotion Emulsion Stability

| | Instability |
|---|---|
| Sample ID | Index |
| Example 1 | 0.002 |
| (1.5 wt % MCG, 0.5 wt % Xanthan Gum) | |
| Example 2 | 0.002 |
| (1.0 wt % MCG, 0.5 wt % Xanthan Gum) | |
| Example 3 | 0.003 |
| (0.75 wt % MCG, 0.5 wt % Xanthan Gum) | |
| Example 4 | 0.003 |
| (0.5 wt % MCG, 0.5 wt % Xanthan Gum) | |
| Example 5 | 0.004 |
| (0.3 wt % MCG, 0.5 wt % Xanthan Gum) | |

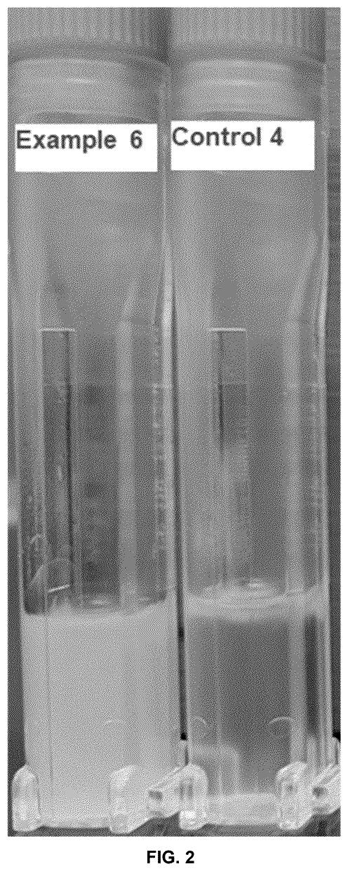

Personal care serums comprising xanthan gum thickener, alone or with MCG as an additional thickener, were tested for emulsion stability (Table 9). The presence of MCG (0.4 wt %) and xanthan gum (0.2 wt %) stabilized oil in the serum, whereas xanthan gum alone as thickener in serum did not stabilize oil. This result is visually shown in FIG. 2.

TABLE 9
Serum Emulsion Stability

| | Instability |
|---|---|
| Sample ID | Index |
| Example 6 | 0.099 |
| (0.4 wt % MCG, 0.2 wt % Xanthan Gum) | |
| Control 4 | 0.511 |
| (0.2 wt % Xanthan Gum) | |

What is claimed is:

1. An aqueous composition comprising xanthan gum and insoluble alpha-glucan, wherein:
(i) at least about 50% of the glycosidic linkages of the insoluble alpha-glucan are alpha-1,3 glycosidic linkages,
(ii) the weight-average degree of polymerization (DPw) of the insoluble alpha-glucan is at least 10, and
(iii) the insoluble alpha-glucan is in the form of particles having a degree of crystallinity of at least about 0.65.

2. The aqueous composition of claim 1, wherein at least about 90% of the glycosidic linkages of the insoluble alpha-glucan are alpha-1,3 glycosidic linkages.

3. The aqueous composition of claim 1, wherein the DPw of the insoluble alpha-glucan is about 15 to 100.

4. The aqueous composition of claim 1, wherein the DPw of the insoluble alpha-glucan is about 35 to about 100.

5. The aqueous composition of claim 1, wherein the DPw of the insoluble alpha-glucan is about 35 to about 60.

6. The aqueous composition of claim 1, wherein the insoluble alpha-glucan particles have a degree of crystallinity of at least about 0.7.

7. The aqueous composition of claim 1, comprising about 0.05% to 5% by weight of the insoluble alpha-glucan.

8. The aqueous composition of claim 7, comprising about 0.05% to 1% by weight of the insoluble alpha-glucan.

9. The aqueous composition of claim 8, comprising about 0.1% to 1% by weight of the insoluble alpha-glucan.

10. The aqueous composition of claim 1, comprising about 0.05% to 2% by weight of the xanthan gum.

11. The aqueous composition of claim 10, comprising about 0.05% to 1% by weight of the xanthan gum.

12. The aqueous composition of claim 1, comprising at least about 50% by weight water.

13. The aqueous composition of claim 12, comprising at least about 70% by weight water.

14. The aqueous composition of claim 1, wherein the composition is a dispersion.

15. The aqueous composition of claim 1, wherein the composition is an emulsion.

16. The aqueous composition of claim 1, wherein the composition is a household care product, personal care product, industrial product, ingestible product, or pharmaceutical product.

17. The aqueous composition of claim 1, wherein the composition is a lotion or serum.

18. The aqueous composition of claim 17, wherein the lotion or serum is a skin care product.

19. The aqueous composition of claim 1, wherein the composition has enhanced stability.

20. The aqueous composition of claim 1, wherein the composition has enhanced viscosity.

21. A method of producing an aqueous composition according to claim 1, said method comprising:
blending together at least water, the xanthan gum, and the insoluble alpha-glucan.
